(19) United States
(12) Patent Application Publication
(10) Pub. No.: US 2005/0271735 A1
(43) Pub. Date: Dec. 8, 2005

(54) Method of encapsulating hydrophobic organic molecules in polyurea capsules

(76) Inventors: Harald D. H. Stover, Dundas (CA); Wen-Hui Li, Mississauga (CA); Lisa M. Croll, Brockville (CA)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/516,060

(22) PCT Filed: Jun. 2, 2003
(86) PCT No.: PCT/CA03/00817

§ 371 Date: Aug. 1, 2005

Related U.S. Application Data
(60) Provisional application No. 60/384,137, filed on May 31, 2002.

(51) Int. Cl. A61K 9/16; A61K 9/50; B01J 13/04

(52) U.S. Cl. 424/490; 264/4.1

(57) ABSTRACT

It is known to encapsulate various materials in polyurea microcapsules, but obtaining satisfactory microcapsules incorporating alcoholic materials has proven difficult. A process has now been found where polyurea microcapsules are formed by interfacial polymerization between an aqueous phase and a water-immiscible phase, and properties, particularly the solubility parameters, of the water immiscible phase are closely matched to corresponding properties of the polyurea. Microcapsules prepared by this process have improved stability, mechanical strength and controlled release properties.

### FIELD OF THE INVENTION

The present invention relates to microcapsules, and to a process for making them.

### BACKGROUND OF THE INVENTION

Microcapsules containing an encapsulated active ingredient are known for many purposes. In the area of crop protection, insect pheromones that are slowly released from microcapsules are proving to be a biorational alternative to conventional hard pesticides. In particular, attractant pheromones can be used effectively in controlling insect populations by disrupting the mating process. Here, small amounts of species-specific pheromone are dispersed over the area of interest during the mating season, raising the background level of pheromone to the point where the male insect cannot identify and follow the plume of attractant pheromone released by his female mate. Alternatively, pheromones may be used as additives in microencapsulated pesticides, in order to help attract specific insects to the microcapsules.

Polymer microcapsules, in particular, serve as efficient delivery vehicles, as they: a) are easily prepared by a number of interfacial and precipitation polymerizations, b) enhance the resistance of the pheromone to oxidation and irradiation during storage and release, c) may in principle be tailored to control the rate of release of the pheromone fill, and (d) permit easy application of pheromones by, for example, spraying, using conventional spraying equipment.

One known method of forming pheromone-filled microcapsules, interfacial polymerization, involves dissolving a pheromone and a diisocyanate or a polyisocyanate in xylene and dispersing this solution into an aqueous solution containing a diamine or a polyamine. A polyurea membrane forms rapidly at the interface between the continuous aqueous phase and the dispersed xylene droplets, resulting in formation of microcapsules containing the pheromone and xylene; see for example PCT international application WO 98/45036 [Sengupta et al., published Oct. 15, 1998].

Although this method is useful and yields valuable products, it does have some limitations. Isocyanates are highly reactive compounds, and it is at times difficult to encapsulate compounds that react with the isocyanate. For example, it is difficult to encapsulate compounds containing hydroxyl groups such as alcohols. Some efforts have succeeded in encapsulating alcohols, as seen, for example, in WO 98/45036. The formed microcapsules, however, lack the stability and mechanical strength desirable for commercial use. This may be due to the chemical reaction between the alcoholic pheromone and the isocyanate, which reaction competes with wall formation and leads to weaker walls. It may also be due to the interfacial activity of the alcoholic pheromone, or the urethane it forms by reaction with isocyanate, interfering with the colloidal stability of the microcapsules.

Accordingly, there still remains a need for a process that encapsulates pheromones, particularly alcohol pheromones, to yield microcapsules that have good storage stability, mechanical strength and controlled release characteristics to permit their successful use in agriculture and horticulture.

### SUMMARY OF THE INVENTION

According to one aspect of the invention there is provided a process for encapsulation of a hydrophobic organic molecule in a polyurea microcapsule by interfacial polymerization, the process comprising contacting
a) an aqueous phase comprising an amine-bearing compound selected from a diamine and a polyamine, and
b) a water-immiscible phase comprising a water-immiscible solvent, an isocyanate-bearing compound selected from a diisocyanate and a polyisocyanate, and a hydrophobic organic molecule
   wherein the water-immiscible solvent has a solubility parameter that is below the solubility parameter of the polyurea microcapsule. This may be achieved by choosing an immiscible phase that has a solubility parameter that is below that of the polyurea and is preferably within the range of about 3-8 Mpa^{1/2} below the solubility parameter of the polyurea, and more preferably within the range of 4-6 Mpa^{1/2} below the solubility parameter of the polyurea. More specifically, and recognizing that solubility parameters are only very rough guides to overall polymer-solvent interaction, this may be achieved by chosing an immiscible phase that may have a solubility parameter outside of this range, but that by virtue of its hydrogen bonding interaction or dipolar nature is still able to slightly swell the polyurea wall.

The most commonly used one-dimensional solubility parameter is the Hildebrand solubility parameter. It has been complemented with three dimensional parameters such as the Hansen solubility parameters, that break the overall substance-solvent interaction into three terms: a dipolar term, a hydrogen-bonding term, and a dispersive term. The dispersive term is considered to be of little influence in the present context, dealing with strongly polar and hydrogen-bonded polyurea, and hence emphasis has been placed on the dipolar and hydrogen-bonding terms of the solvents. Examples of these solubility parameters are given in Table 1 below.

Polyurea moities, when formed, display hydrogen bonding. A solvent that is capable of engaging in hydrogen bonding will cause some solvent-polyurea hydrogen bonding, thereby interfering to some extent with polyurea-polyurea hydrogen bonding and causing swelling of the polyurea.

As well, a permeable polyurea capsule wall may be achieved by choosing an immiscible fill that may have a solubility parameter more than approximately 7 Mpa^{1/2} lower than the polyurea, does not engage in strong hydrogen bonding or dipolar interactions with polyurea, but is polar enough to permit rapid and effective partitioning of the second, aqueous wall forming component, usually a di- or oligoamine, across the interface and into the immiscible phase. Butyl acetate is an example of such a solvent.

The immiscible phase has to be chosen so as to combine the properties of hydrogen bonding and polarity, in order to provide an interfacial system wherein the aqueous amine can rapidly and quantitatively partition into the immiscible organic phase, throughout the period needed for conversion of the isocyanate.

In other words, in order for the amine to compete effectively with the alcoholic pheromone for reaction with an isocyanate, the amine should not be stopped by a dense, diffusion-limiting polyurea skin. An immiscible phase chosen to swell the polyurea wall will typically also have a fairly high affinity for the amine, and hence facilitate partitioning of the amine.

Upper limits to the desirable solubility parameters of the encapsulation solvents are given by the increasing miscibility of the solvent phase with water, as well as by the decreasing ability of the immiscible phase to dissolve the hydrophobic fill. For example, as described below, dimethylphthalate (DMP), with a solubility parameter of approximately 22 MPa^{1/2}, under certain conditions absorbs sufficient water to become a poor solvent for the hydrophobic dodecanol. DMP can be used as immiscible phase provided a less polar co-solvent such as xylene is added to reduce the overall solubility parameter of the resulting solvent mixture.

The invention also extends to a microcapsule comprising a water-immiscible solvent and a hydrophobic organic molecule, encapsulated by a polyurea microcapsule which is swollen by the water-immiscible solvent. By means of the invention it is possible to prepare microcapsules that encapsulate alcohol in amounts of 5% or greater, based on the weight of the water-immiscible phase. Examples below show microcapsules made by the process of the invention that have a pheromone loading of 10%, 20% and 30%, based on the weight of the water-immiscible phase, and that release the pheromone over periods of sixty days or more. Stability and controlled release over this period of time is adequate for control of insect populations, as it approximately equates to the mating season of insects.

The invention also extends to the formation of polyurea capsules containing fills other than alcoholic pheromones, wherein choosing a solvent phase with a solubility parameter as close as feasible to that of the polyurea capsule wall lead to rapid and quantitative formation of capsule walls, that are swollen by the solvent and hence release their fill readily.

In another aspect, the invention provides the use of a microcapsule, as described above, for the controlled release of a volatile hydrophobic organic molecule.

### DESCRIPTION OF THE FIGURES

Specific embodiments of the invention are further described with reference to the attached Figures, of which:

FIG. 1 shows the weight loss of polyurea (PU) capsules formed from Mondur ML and diethylenetriamine (DETA) with different solvents in absence of 1-dodecanol.

FIG. 2 shows optical micrographs of the polyurea microcapsules formed from Mondur ML and DETA, with 20% 1-dodecanol, and 80% solvent in the core. The size bar applies to all four images. The solvents were butyl acetate (BuAc), propyl acetate (PrAc), butyl benzoate (BuBz) and ethyl benzoate (EtBz).

FIG. 3 shows optical micrographs of polyurea microcapsules formed from Mondur ML and DETA, with 10% 1-dodecanol and 90% solvents in the core, after storage in aqueous suspension for about six months.

FIG. 4 shows typical Environmental Scanning Electron Microscopy (ESEM) and Transmission Electron Microscopy (TEM) images for polyurea microcapsules formed from Mondur ML and DETA, with 20% 1-dodecanol and 80% butyl benzoate in the core.

FIG. 5 graphs the effect of single solvents on the release from polyurea capsules formed from Mondur ML-DETA with 20% 1-dodecanol and 80% solvent in the core.

FIG. 6 graphs the effect of co-solvent composition on release from polyurea capsules formed from Mondur ML-DETA, with 10% 1-dodecanol and 90% total cosolvent in the core.

FIG. 7 graphs the effect of co-solvents on the release from polyurea capsules formed from Mondur ML and DETA, with 20% 1-dodecanol and 80% solvent or co-solvents.

FIG. 8 graphs the effect of crosslinking on polyurea capsules formed from Mondur ML and Mondur MRS, and DETA and tetraethylenepentamine (TEPA), respectively, with 20% 1-dodecanol and 80% BuBz.

FIG. 9 graphs the effect of 1-dodecanol loading on the release of polyurea capsules formed from Mondur ML and TEPA with BuBz as solvent. Mondur ML loading: 2.5%.

FIG. 10 graphs the effect of isocyanate loading on the release from polyurea capsules formed from Mondur ML and DETA, with 20% 1-dodecanol and 80% BuBz. Mondur ML loading: 2.5%

FIG. 11 shows optical micrographs of polyurea microcapsules formed from Mondur MRS and TEPA, and using 20 mL 1-dodecanol, 40 mL isopropyl myristate and 40 mL methyl isoamyl ketone (MIAK) as the oil phase.

FIG. 12 shows a transmission electron micrograph (TEM) of the polyurea capsules formed from Mondur ML and DETA, using 20% 1-dodecanol and 80% isopropyl myristate for the organic phase.

FIG. 13 shows the results of observations of release rates from polyurea capsules described in FIG. 12, formed with 20% 1-dodecanol and 80% isopropyl myristate and using Mondur ML and DETA.

FIG. 14 illustrates how the in-diffusing amine and oil-borne hydroxy-functional pheromone compete for the available isocyanate in each forming capsule.

### DESCRIPTION OF PREFERRED EMBODIMENTS

The solubility parameter of substances can be used to indicate the miscibility of the substances; the closer the values of the solubility parameter of two substances the more miscible they generally will be. In the case of one of these substances being a crosslinked polymer and the other being a solvent, it is typically found that the closer the solubility parameters of these two substances, the more the polymer will be swollen by the solvent. It has been found that by matching the solubility parameter of the water-immiscible liquid to the solubility parameter of the crosslinked polyurea that forms the wall of the microcapsule, within the upper limits described above, there can be obtained microcapsules of enhanced stability and mechanical strength and improved controlled release characteristics. Polyurea formed from aromatic isocyanates typically has a solubility parameter of approximately 25 Mpa^{1/2}. This high value of the solubility parameter is in large part due to the strong internal hydrogen bonding characteristic of urea compounds in general.

To prevent formation of a diffusion-limiting polyurea skin at the interface requires either a strong hydrogen bonding solvent to swell the polyurea, or a polar solvent with a high affinity towards the amine to facilitate its in-diffusion. Good hydrogen-bonding properties and high polarity often go hand-in-hand, and are also highly correlated with the solubility parameter, as well. Since solubility parameters are known for many solvents, this parameter is used here as one criterion to describe the choice of immiscible phase. It is however not meant to be an exclusive criterion, for the reasons given above.

A suitable water immiscible liquid often has a value of solubility parameter about 3-8 Mpa^{1/2} below the solubility parameter of the polyurea, preferably about 4-6 Mpa^{1/2} below the solubility parameter of the polyurea.

The water-immiscible phase is a mixture of substances containing at least a water-immiscible solvent, a material to be encapsulated such as a hydrophobic pheromone, in particular hydrophobic pheromones containing an alcohol group, and a di- or polyisocyanate, and possibly also one or more co-solvents. The solubility parameter of interest is the solubility parameter of this mixture. The closer that this equates to the solubility parameter of the polyurea, while still remaining immiscible with water, and able to dissolve the hydrophobic fill, the better the results obtained, in general.

The solubility parameter of a particular polyurea will depend upon the particular polyisocyanate and polyamine from which it is formed. Due to their strong hydrogen bonding ability, and few applications requiring solvent swelling, the solubililty parameters of polyureas have not been routinely measured. They are known to be around 25 Mpa^{1/2} for aromatic polyureas. It is likely that they may be lowered by introducing aliphatic isocyanates, and by incorporating longer spacers between urea linkages. In some preferred embodiments, therefore, a selected isocyanate is reacted with a selected polyamine to form a polyurea, the value of the solubility parameter of the formed polyurea is determined, for example by measuring the physical degree of swelling in a number of solvents covering a range of solubility parameters. This value is used as a guide in determining the solubility parameter, and therefore the composition of the water immiscible liquid that is used in the interfacial polymerization.

The properties of the organic phase are adjusted in terms of polarity and hydrogen bonding ability, to facilitate reaction of the isocyanate with the amine and to reduce interference from the alcohol when using an alcoholic fill. Thus, the composition of the organic phase is adjusted to enhance or maximize the rate and completeness of wall formation, and to achieve control of release rates of both solvent and fill. In addition, the release rates of solvent and fill can be controlled through the choice of crosslinking agents.

The solvents that have been commonly used as organic phase in the prior art, namely, xylene and toluene, are in general not sufficiently polar for encapsulation of hydroxyl-functional pheromones in the most commonly used, aromatic polyureas. It is preferred to use non-reactive liquids that have higher polarity and solubility parameters, and mention is made of aliphatic and aromatic mono- and diesters, especially the C₁-C₁₂ alkyl esters of acetic, propionic, succinic, adipic, benzoic and phthalic acid. For esters of aliphatic acids or for esters of aromatic acids, it is preferred that the alkyl moiety has from 1 to 8 carbon atoms. In either case, the alkyl group may be linear or branched. With di-acids, the alkyl moieties may be the same or different. Similarly, alkyl esters of longer chain aliphatic acids are suitable, such as isopropyl tetradecanoate, also called isopropyl myristate. It is possible for the esters to bear additional substituents, for example alkyl, alkoxy, alkoxyalkyl and alkoxyalkoxy, containing up to 8 carbon atoms.

Suitable solvents also may include esters of ethylene glycol and glycerol, in particular glyceryl triacetate, glyceryl tripropionate, glyceryl tributyrate, and higher triglycerides, as well as acetyl triethyl citrate. Mention is also made of ketones such as methyl isobutyl ketone, methyl tert.-butyl ketone, methyl amyl ketone, methyl isoamyl ketone and other ketones having up to 12 carbon atoms. These solvents may be used alone or in admixture with each other or in admixture with other non-polar solvents, for example aromatic solvents such as toluene and xylene, alicyclic solvents such as cyclohexane, and commercially available hydrocarbon solvents.

Properties of some organic liquids, and or polyurea are given below:

TABLE 1

| | | | Hydrogen | |
|---|---|---|---|---|
| | Hildebrand | Polar | Bonding | |
| | Solubility | Parameter | Parameter | Boiling |
| | Parameter | δₚ | δₕ | Point |
| Solvent | δ [Mpa^{1/2}] | [Mpa^{1/2}] | [Mpa^{1/2}] | [° C.] |
| Xylenes | p-: 18.0^{a} | o: 1^{a} | o-: 3.1^{a} | 137-144 |
| | m-: 18.2^{b} | m: 7.2^{b} | m-: 2.4^{b} | |
| | o-: 18.5^{b} | o: 7.5^{b} | o-: 0.0^{b} | |
| | p-: 18.1^{b} | p: 7.0^{b} | p-: 2.2^{b} | |
| butyl benzoate | 19.4^{b} | 9.4^{b} | 5.9^{b} | 249 |
| butyl acetate | 17.4^{a} | 3.7^{a} | 6.3^{a} | 124-126 |
| | 17.8^{b} | 7.8^{b} | 6.8^{b} | |
| Dimethyl | 21.9^{a} | 10.8^{a} | 4.9^{a} | 282 |
| phthalate | 22.5^{b} | 12.6^{b} | 9.7^{b} | |
| Isopropyl | | | | 320 |
| myristate | | | | |
| Isopropyl | 15.3^{a} | 3.9^{a} | 3.7^{a} | |
| palmitate | | | | |
| Triacetin | 22.0^{b} | 11.6^{b} | 11.2^{b} | 258-260 |
| Methyl amyl | 18.4^{b} | 7.6^{b} | 7.2^{b} | 151.5 |
| ketone | | | | |
| Methyl isoamyl | 17.4^{a} | 5.7^{a} | 4.1^{a} | 142-145 |
| ketone | | | | |
| Urea- | 25.74^{a} | 8.29^{a} | 12.71^{a} | |
| formaldehyde | | | | |
| resin | | | | |
| (Plastopal H, | | | | |
| BASF) | | | | |
| 1,1,3,3- | 21.7^{a} | 8.2^{a} | 11^{a} | |
| tetramethylurea | | | | |
| Polyurea^{c} | ˜25 | | (high) | |

^{a}Polymer Handbook, 4^{th} Ed., Brandrup & Immergut
^{b}CRC Handbook of Solubility Parameters and Other Cohesion Parameters, Allan, Barton, CRC Press 1983.
^{c}Ryan, A. J.; Stanford, J. L, ; Still, R. H. Polym. Commun. 29(1988), 196.

Desirably, the first liquid is a solvent that will swell the forming polyurea wall. For ease of handling, it should preferably have a boiling point in the vicinity of 100° C., or higher. The properties of the first liquid, which will become encapsulated with the active material that is to be released, will affect the rate of wall formation and the rate of release of that active material. Selection of a first liquid has to be made with these considerations in mind.

Suitable candidates for use as the first liquid include alkylbenzenes such as toluene and xylene (provided a polar cosolvent is added to enhance their polarity), halogenated aliphatic hydrocarbons such as dichloromethane, aliphatic nitriles such as propionitrile and butyronitrile, ethers such as methyl tert.-butyl ether, linear and branched ketones such as methylisobutylketone and methyl amyl ketone, esters such as ethyl acetate and higher acetates (preferably propyl acetate), as well as the analogous propionates, benzoates, adipates and phthalates, and esters of glycerol with acetic, propionic and butyric acid.

Mixtures of solvents can be used. There can also be used co-solvents to change the solubility parameter of the solvents or solvent mixtures, particularly their polarity and their hydrogen bonding ability. As co-solvents there are mentioned aliphatic liquids such as kerosene, alicyclic hydrocarbons such as cyclohexane, and hydrophobic esters such as isopropyl myristate or methyl myristate.

As stated above, xylenes and toluene are insufficiently polar to be used as the only solvent with a long-chain alcohol that is to be encapsulated. It is possible for a solvent to be too polar to be satisfactorily used, and dimethyl phthalate (DMP) is such a solvent. In the case of encapsulation of long-chain alcohols such as dodecanol in polyurea formed from aromatic isocyanates and short polyamines such as DETA or TEPA for example, it is preferred that the polarity of the water-immiscible liquid is greater than that of xylenes and toluene, but less than that of DMP. It is possible to use xylenes and toluene as solvent, in admixture with one or more co-solvents such as DMP, or aliphatic esters that enhance its polarity. It is possible to use DMP as solvent, in admixture with one or more co-solvents that reduce its polarity. Similar considerations apply to the use of polar esters such as glycerol triacetate, and related polar low molecular weight citric acid esters.

For good release characteristics, it is desirable that the organic solvent and the hydrophobic active fill shall have the same, or similar, boiling points. It is therefore preferred that the organic solvent and the hydrophobic fill shall have boiling points that are not more than about 50° C. apart, and it is particularly preferred that they shall not be more than about 20° C. apart. This leads to facilitated transport through the capsule wall, with the solvent component helping to swell the polyurea wall and facilitating release of the active fill.

Alternatively, low boiling solvents such as propyl acetate, butyl acetate or methyl isoamyl ketone may be used as well. Here, the solvent vaporizes rapidly within the first few hours of release, to be followed by a slower release of the less volatile fill. This situation is acceptable in case of liquid, non-viscous fills, but less desirable in the case of fills that may crystallize upon loss of solvent from the core.

The continuous phase is preferably water or an aqueous solution with water as the major component.

The polyisocyanate may be a diisocyanate, a triisocyanate, or an oligomer. The polyisocyanate may be aromatic or aliphatic and may contain two, three or more isocyanate groups. Examples of aromatic polyisocyanates include 2,4- and 2,6-toluene diisocyanate, naphthalene diisocyanate, diphenylmethane diisocyanate (Mondur ML), and triphenylmethane-p,p′,p″-trityl triisocyanate.

Aliphatic polyisocyanates may optionally be selected from aliphatic polyisocyanates containing two isocyanate functionalities, three isocyanate functionalities, or more than three isocyanate functionalities, or mixtures of these polyisocyanates. Preferably, the aliphatic polyisocyanate contains 5 to 30 carbons. More preferably, the aliphatic polyisocyanate comprises one or more cycloalkyl moieties. Examples of preferred isocyanates include dicyclohexylmethane-4,4′-diisocyanate; hexamethylene 1,6-diisocyanate; isophorone diisocyanate; trimethyl-hexamethylene diisocyanate; trimer of hexamethylene 1,6-diisocyanate; trimer of isophorone diisocyanate; 1,4-cyclohexane diisocyanate; 1,4-(dimethylisocyanato) cyclohexane; biuret of hexamethylene diisocyanate; urea of hexamethylene diisocyanate; trimethylenediisocyanate; propylene-1,2-diisocyanate; and butylene-1,2-diisocyanate. Mixtures of polyisocyanates can be used.

Particularly preferred polyisocyanates are polymethylene polyphenylisocyanates of formula: 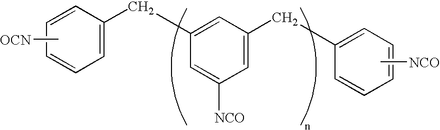
wherein n is from 0-4. These compounds are available under the trade-mark Mondur, with Mondur ML being the compound in which n is 0 and Mondur MRS being a mixture of compounds of which n typically is in the range from 0 to 4.

Suitable reactants that will react with isocyanates include water-soluble primary and secondary polyamines, preferably primary diamines. These include diamines of formula (I):
H₂N(CH₂)ₙNH₂ (I)
wherein n is an integer from 2 to 10, preferably 2 to 6.

Also suitable are mixed primary/secondary amines, and mixed primary/secondary/tertiary amines. Mixed primary/secondary amines include those of Formula (II): 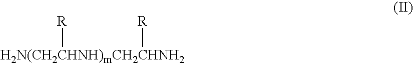
wherein m is an integer from 1 to 1,000, preferably 1 to 10 and R is hydrogen or a methyl or ethyl group. Mention is made of diethylene triamine (DETA), tetraethylene pentamine (TEPA), and hexamethylenediamine (HMDA). Suitable primary/secondary/tertiary amines include compounds like those of formula (II), but modified in that one or more of the hydrogen atoms attached to non-terminal nitrogen atoms of the compound of formula (II) is replaced by a lower aminoalkyl group such as an aminoethyl group. The commercial product of tetraethylenepentamine usually contains some isomers branched at non-terminal nitrogen atoms, so that the molecule contains one or more tertiary amino groups. All these polyamines are readily soluble in water, which is suitable for use as the aqueous continuous phase. Other suitable polyamine reactants include polyvinylamine, polyethyleneimine, polypropyleneimine, and polyallylamine.

Primary and secondary amino groups will react with isocyanate moieties. Tertiary amino groups catalyse the reaction of the primary and secondary amino groups, as well as the conversion of isocyanate groups into amine groups that can subsequently react further with additional isocyanate groups.

Also suitable are polyetheramines of general formula (III): 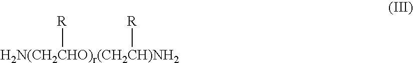
where r is an integer from 1 to 20, preferably 2 to 15, more preferably 2 to 10, and R is hydrogen, methyl or ethyl. Such compounds, as well as their analogues based on propyleneoxide repeat units, are available under the trademark Jeffamine from Huntsman.

To be useful as a reactant and not merely as a catalyst, the amine must contain at least two primary or secondary amino groups. Hence, the compound must be, at least, a diamine, but it may contain more than two amino groups; see for example compounds of formula (II). In this specification the term “diamine” is used to indicate a compound that has at least two reactive amino groups, but the term does not necessarily exclude reactants that contain more than two amino groups.

The pheromone or other material that is to be encapsulated in the microcapsules is dissolved or dispersed in the solution with the isocyanate. As indicated above, this material must not be so reactive with the isocyanate that it competes significantly with the reaction that creates the membrane. Although alcohols will react with isocyanate moieties to form urethanes, these reactions are relatively slow, compared with the reactions between the isocyanate moiety and the amine, so these reactions do not compete significantly with the desired membrane-forming reactions, provided the polyurea formation is fast. It is an aspect of this invention to teach conditions where the wall-forming reaction of the amine with the polyisocyanate is of the same order, or faster, than the competing reaction of alcoholic fills with the polyisocyanates. As stated above, the rate of the membrane-forming reaction depends on the particular liquid that is used as the dispersed organic phase.

A catalyst can be incorporated with the amine in the aqueous phase to speed the membrane-forming reactions. Suitable catalysts include tertiary amines. The tertiary amine catalyst, in the amount used, should be freely soluble in the water present in the reaction mixture. The simplest tertiary amine is trimethylamine and this compound, and its C₂, C₃ and C₄ homologues can be used. It is of course possible to use tertiary amines containing a mixture of alkyl groups, for instance methyldiethylamine. The tertiary amine can contain more than one tertiary amine moiety.

The tertiary amine may also contain other functional groups provided that those other functional groups do not interfere with the required reaction, or the functional groups participate beneficially in the required reaction. As an example of a functional group that does not interfere there is mentioned an ether group. As examples of groups that participate there are mentioned primary and secondary amino groups, and hydroxyl groups. Examples of suitable tertiary amines include compounds of the following structures: 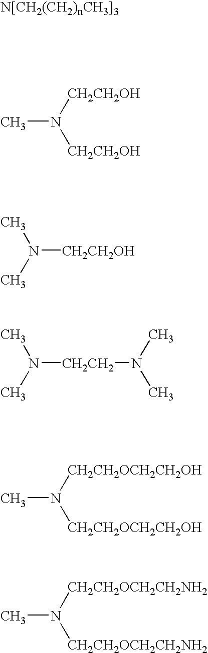
Of the tertiary amines, triethylamine (TEA) is preferred.

The amount of the tertiary amine required is not very great. It is conveniently added in the form of a solution containing 0.5 g of TEA per 10 mL of water. Usually 0.5% by weight of this solution, based on the total weight, suffices, although 0.7% may be required in some cases. The amount used does not usually exceed 1%, although no disadvantage arises if more than 1% is used.

Catalysts other than tertiary amines can be used. Metal salts that are soluble in an organic solvent used as the first liquid can be used. Mention is made of titanium tetraalkoxides available under the trademark Tyzor from DuPont and stannous octanoate, although these should not be used when there is also present in the organic solvent an alcohol to be encapsulated.

The ability to encapsulate alcohols is of particular significance. The key component of the pheromone of the codling moth is E,E-8,10-C₁₂ alcohol and it has been difficult to encapsulate this pheromone by the previously known technique involving isocyanate. The present invention permits encapsulation of alcoholic pheromones, and provides long term storage stability, handling stability and controlled release.

The liquid that serves as the dispersed phase, is a liquid in which the isocyanate can be dispersed or dissolved and in which the pheromone to be encapsulated can be dispersed or dissolved. The liquid should be immiscible, or at least only partially miscible, with the aqueous phase. While the limits on what is meant by “partially miscible” are not precise, in general a substance is considered to be water-immiscible if its solubility in water is less than about 0.5% by weight. It is considered to be water-soluble if its solubility is greater than 98%, i.e., when 1 gram of the substance is put in 100 grams of water, 0.98 gram would dissolve. A substance whose solubility falls between these approximate limits is considered to be partially water-miscible. An example of a partly miscible solvent is glycerol triacetate, which is soluble in 14 parts water.

Surfactants and stabilizers can be used to assist in dispersion of organic, or oil, phase in the aqueous liquid. Mention is made of stabilizers such as poly(vinylalcohol), polyvinylpyrrolidones, Methocel and surfactants such as polyoxyethylene(20) sorbitan monooleate, available under the trademark Tween 80. Other suitable surfactants and emulsifiers include polyethyleneglycol alkyl ethers, for example C₁₈H₃₅(OCH₂CH₂)ₙOH, where n has an approximate value of about 20, available under the trade-mark BRIJ 98, or nonylphenyl-oligo-ethylene glycol, available under the trademark IGEPAL.

Ionic surfactants can be used. Sodium dodecyl sulphate (SDS) is mentioned as an example of an anionic surfactant.

The organic liquid can be dispersed in the aqueous liquid by dropping the organic liquid into a stirred bath of the aqueous liquid. The organic liquid then forms droplets throughout the continuous phase of the aqueous liquid. The amine may be present in the aqueous liquid before the organic liquid is added. In an alternative, and preferred embodiment, the amine is not present in the aqueous liquid when the organic liquid is being dispersed, but is added subsequently. In any event, the reactants meet and react near the interface between the continuous and dispersed phases, that is, near the surface of the droplets, and react to form the membrane. Specifically, the amine, being the more amphiphilic of the two reactants, is usually considered to cross the interface and partition into the organic fill phase, where it reacts with the isocyanate. Hence one consideration in the present invention is to provide conditions under which the amine can efficiently partition into the organic fill phase and hence successfully compete with the alcoholic pheromone in reaction with the isocyanate.

The membrane-forming reaction can be carried out at a temperature above 0° C., at room temperature or at elevated temperature. Usually, lower temperatures such as room temperature, are preferred in the present invention, in order to minimize the undesired side reaction between isocyanate and alcoholic pheromone. If elevated temperatures are used, the optimum temperature will also depend on the boiling point of each of the solvents that make up the dispersed and continuous phases and that of the material to be encapsulated. No advantage is seen in using a temperature greater than about 70° C. No advantage is anticipated in carrying out the reaction at temperatures below 0° C., in presence of freezing point depressing additives to the aqueous phase.

When microcapsules are formed from a first liquid having a density less than that of water, they will usually rise and gather at the top of the liquid present. They can be shipped in this form, or concentrated by decantation.

As examples of materials to be encapsulated, particular mention is made of compounds such as insect pheromones. Pheromones containing hydroxyl groups, i.e., alcohols, are of particular interest. These are compounds typically containing from 8 to 20 carbon atoms and at least one hydroxyl group, usually a primary hydroxyl group, but sometimes secondary or tertiary. They may be mono- or polyunsaturated and may also contain a further functional group or groups, for example an epoxy, aldehydic or ester group. A compound that is a significant component of several insect pheromones, and is a useful model for other pheromones in experiments, is dodecan-1-ol.

In the notation used herein to describe the structure of the pheromones, the type (E or Z) and position of the double bond or bonds are given first, the number of carbon atoms in the chain is given next and the nature of the end group is given last. To illustrate, the pheromone Z-10 C19 aldehyde has the structure:

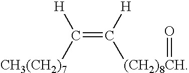

Pheromones may in fact be mixtures of compounds with one component of the mixture predominating, or at least being a significant component. Mentioned as examples of significant or predominant components of insect pheromones, with the target species in brackets, are the following: E/Z-11 C14 aldehyde (Eastern Spruce Budworm), Z-10 C19 aldehyde (Yellow Headed Spruce Sawfly), Z-11 C14 acetate (Oblique Banded Leafroller), Z-8 C12 acetate (Oriental Fruit moth) and E,E-8,10 C12 alcohol (Codling moth).

An example of a ketone that is a pheromone is E or Z 7-tetradecen-2-one, which is effective with the oriental beetle. An ether that is not a pheromone but is of value is 4-allylanisole, which can be used to render pine trees unattractive to the Southern pine beetle.

As indicated, the invention is particularly useful for encapsulating alcohols, and mention is made of 1-dodecanol and mono- and di-unsaturated alcohols, for example E-11-tetradecen-1-ol, Z-11 C₁₄ alcohol, Z-8 C₁₂ alcohol and E,E-8,10 dodecadiene-1-ol alcohol. The invention is also useful for encapsulating other pheromones such as those containing ketone, aldehyde or ester groups, as the strong yet permeable capsule wall formed in presence of suitable polar and hydrogen-bonding solvents will give desirable linear release profiles.

The amount of active fill incorporated in the microcapsules can be up to 30% by weight, based on the total weight of the water-immiscible phase. For distributing pheromones for controlled release it is often desirable that the microcapsule loading shell be as high as possible. In the present invention, using alcoholic pheromones, the undesired side reaction between the pheromone and the isocyanate would increase with increasing pheromone loading. Successful pheromone loadings of 30% have been achieved, as demonstrated below.

In one preferred embodiment, the product of the microencapsulation process is a plurality of microcapsules having a size in the range of from about 1 to about 2000 μm, preferably 10 μm to 500 μm. Particularly preferred microcapsules have sizes in the range from about 10 μm to about 60 μm, more preferably about 20 to about 30 μm, and an encapsulated pheromone contained within the capsule membrane. The microcapsules can be used in suspension in water to give a suspension suitable for aerial spraying. The suspension may contain a suspending agent, for instance a gum suspending agent such as guar gum, rhamsan gum or xanthan gum.

Incorporation of a light stabilizer, if needed to protect the encapsulated material, is within the scope of the invention. Suitable light stabilizers include the tertiary phenylene diamine compounds disclosed in Canadian Patent No. 1,179,682, the disclosure of which is incorporated by reference. The light stabilizer can be incorporated by dissolving it, with the pheromone, in the organic phase. Antioxidants and UV absorbers can also be incorporated. Many hindered phenols are known for this purpose. Mention is made of antioxidants available from Ciba-Geigy under the trademarks Irganox 1010 and 1076. As UV absorbers there are mentioned Tinuvin 292, 400, 123 and 323 available from Ciba-Geigy.

To assist in determining the distribution of sprayed microcapsules it is possible to include a coloured dye or pigment in the microcapsules. The dye should be oil-soluble and can be incorporated, with the pheromone, in the oil phase. It should be used only in a small amount and should not significantly affect the membrane-forming reaction. Alternatively, or additionally, an oil-soluble or oil-dispersible dye can be included in the aqueous suspension of microcapsules, where it is absorbed by the microcapsule shell. Suitable oil-soluble or oil-dispersible dyes can be obtained from DayGlo Color Corporation, Cleveland, Ohio, and include Blaze Orange, Saturn Yellow, Aurora Pink, and the like.

Although the invention has been described largely with reference to encapsulation of pheromones, other molecules that are active in nature can be encapsulated in a similar manner. As examples there are mentioned linalool, terpineol, fenchone, and keto-acids and hydroxy-decenoic acids, which encourage activity of worker bees. Encapsulated 4-allylanisole can be used to make pine trees unattractive to the Southern pine beetle. Encapsulated 7,8-epoxy-2-methyloctadecane can be used to combat the nun moth or the gypsy moth.

Other compounds of interest for encapsulation include mercaptans. Some animals mark territory by means of urine, to discourage other animals from entering that territory. Examples of such animals include preying animals such as wolves, lions, dogs, etc. Ingredients in the urine of such animals include mercaptans. By dispersing microcapsules containing the appropriate mercaptans, it is possible to define a territory and discourage particular animals from entering that territory. For example, the urine of a wolf includes a mercaptan, and distribution of microcapsules from which this mercaptan is gradually released to define a territory will discourage deer from entering that territory. Other materials that can be encapsulated and used to discourage approach of animals include essences of garlic, putrescent eggs and capsaicin.

Other compounds that can be included in the microcapsules of the invention include perfumes, pharmaceuticals, fragrances, flavouring agents and the like.

It is also possible to encapsulate materials for uses other than in nature. Mention is made of dyes, inks, adhesives and reactive materials that must be contained until they are to be used, for instance, by controlled release from a microcapsule or by rupture of a microcapsule.

Other materials that can be encapsulated are mentioned in PCT international application WO 98/45036 mentioned above, the disclosure of which is incorporated herein by reference.

All these applications, and microcapsules containing these materials, are within the scope of the present invention.

The following examples are offered by way of illustration and not by way of limitation.

### EXAMPLES

Formation of Polyurea Capsules by Interfacial Polyaddition

Polyurea (PU) capsules were prepared in a 1 L stirred tank reactor at room temperature. In a typical experiment, 100 ml organic solvent containing 2.5 g (10 mmol) Mondur ML was added to 250 ml distilled water in the reactor. After 5 minutes of mixing at about 400 rpm, 1.03 g (20 mmol) diethylene triamine (DETA) dissolved in 50 mL water was added into the reactor. The aqueous phase contained 0.3 g polyvinyl alcohol (PVA) and/or Tween 80 as a stabilizer or surfactant, respectively. The reaction was continued for about 4 hours, except where indicted otherwise, and the capsule suspensions were transferred into bottles.

Characterization

An Olympus BH-2 optical microscope (OM) was used to observe the appearance of capsules when they were wet, and during drying. The morphologies of the capsules were studied with an ElectroScan 2020 Environmental Scanning Electron Microscopy (ESEM) and a JEOL 1200EX Transmission Electron Microscope (TEM).

Release Measurement

Release of the core material was measured by gravimetry. Aluminium weighing dishes treated with sodium carbonate solution were typically used as the support for the capsules. Mylar film was used for some of the measurements. About 1 mL of capsule-water suspension was spread on the support in such a way as to form a single layer of capsule if possible. These aluminium dishes were placed in a fume hood at ambient temperature, and the weight of the capsules was measured on a precise balance until it remained unchanged.

Yield measurement of polyurea-solvent capsules in absence of active fill.

An aliquot of capsule suspension was filtered under vacuum using a pre-weighed filter paper, and washed three times with water. The dried capsules were transferred to a mortar and ground under liquid nitrogen. The broken capsules were then transferred back on to the same filter paper, washed three times with xylenes, and transferred together with the filter paper to a dish. These capsule walls were dried at 50° C., and weighed, and the yield calculated based on theoretical 100% conversion.

Results and Discussion:

1. % Yield of PU walls formed at ambient temperature from different solvents containing 2.5% Mondur ML, unless otherwise specified:

| | Reaction Time | | |
|---|---|---|---|
| | 4 | 24 | 70 |
| | hours | hours | hours |
| PU(xylenes) | 2.5% | 5% | 6.5% |
| PU(xylenes) (for 25% | 0.6% | 2% | 9.5% |
| Mondur ML loading) | | | |
| PU(DMP) | 88% | | |
| PU(BuBz) | 11% | | |
| PU(BuAc) | 36% | | |

The interfacial reaction takes place near the interface, more specifically, on the organic side of the interface. This polyurea formation is a very fast reaction, the two building materials reacting immediately on contact. Once the primary polyurea wall forms, the subsequent reaction rate, especially in the case of a poor solvent for polyurea, largely depends on the continued diffusion of the amine into the organic phase. More specifically, reaction kinetics may change from largely thermodynamic control (amine partitioning into the organic phase), to include diffusion effects (amine diffusing through the formed polyurea skin). Both partitioning and diffusion through the capsule wall are closely related to the solvent properties and solvent-polymer interactions. Higher solvent polarity favors amine partitioning, and a solvent with a solubility parameter similar to that of polyurea will swell the forming walls, resulting in better permeability of the polymer walls for both amine in-diffusion, and potentially, fill release.

The yield results shown above for model capsules not containing 1-dodecanol reflect the rate of reaction. When using xylenes as a solvent, all the yields were low even for extended reaction time. This may be attributed to both the lower amine partitioning into this non-polar solvent, and to the increased resistance to amine diffusion through the dense polyurea walls formed. Polyurea is likely to form dense walls in xylenes, due to their poorer solvency/affinity for the forming polymer. The resistance to amine diffusion increases significantly as the polymer walls grow. That explains the slower increase of the yield with reaction time.

The highest yields were found with DMP as a solvent. Likely the ester groups of DMP favor amine partitioning, and the relatively similar solubility parameters of DMP and polyurea would cause PU wall swelling and hence further facilitate amine diffusion. It has to be noted that DMP and xylenes are a suitable solvent for the formation of microcapsules only in the absence of 1-dodecanol. In the presence of 1-dodecanol, it is observed that the formed capsules are not stable in suspension but rather aggregate rapidly.

The lower yield observed with BuBz compared to BuAc, is most likely due to the lower amine partitioning in the less polar BuBz, as well as to the higher viscosity of the BuBz.

The microcapsules formed from Mondur ML and DETA, at 2.5% Mondur loading in xylenes, butylacetate, butyl benzoate and dimethylphthalate, after a reaction time of 4 hours at room temperature, showed good spherical shape in the wet state by environmental scanning electron microscopy (ESEM). The microcapsules formed using xylenes (a mixture of o, m and p) as solvent showed well defined polyurea walls, even though the yield was low and the walls were thin, as revealed by transmission electron microscopy (TEM).

The microcapsules formed with dimethyl phthalate (DMP), butyl benzoate (BuBz) and butyl acetate (BuAc) showed thicker, stronger walls, with some fluffy material found on the inner side of the wall, suggesting that the ingress of the amine into the organic phase during wall formation had been rapid, at least at some stages of the reaction.

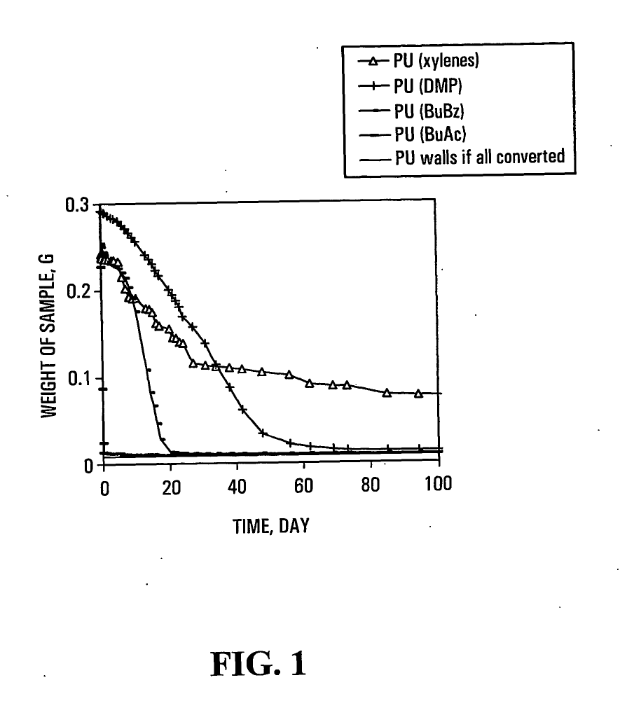

FIG. 1 shows results of observations of release rates from these microcapsules. The microcapsules were formed using Mondur ML at 2.5% loading and DETA, in the absence of 1-dodecanol.

PU(BuAc): very fast release, complete in a few hours. No indication of resistance for BuAc to diffuse out through the polyurea walls, and BuAc evaporated very fast due to its high volatility.

PU(BuBz): fast release, complete in a few days. Again, no indication of resistance for BuBz to diffuse out through the polyurea walls. The higher boiling point of BuBz needs longer time for its evaporation.

PU(DMP): moderate release, complete in about two months, nearly linear. The low volatility of DMP may contribute to the longer release period of this solvent.

PU(xylenes): release rate changes from fast to slow after ˜65% release, and almost stops while release is still incomplete. This slow release may be attributed to diffusion-limited release.

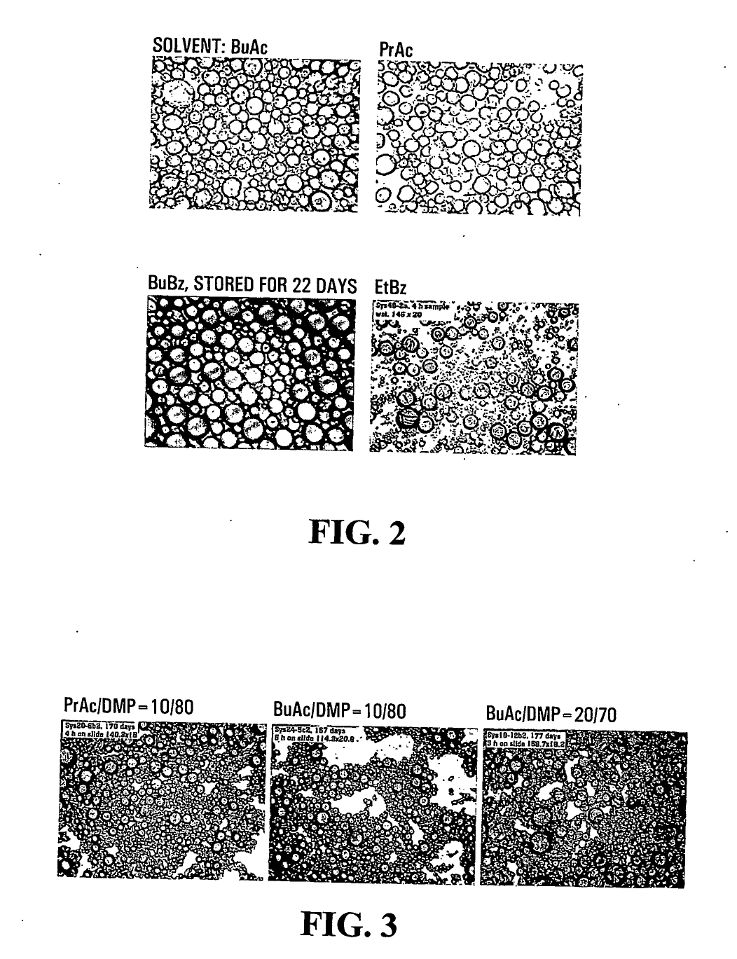

FIG. 2 shows optical microscopy images of microcapsules formed from Mondur ML and DETA, with 20% 1-doecanol and 80% of butyl acetate, propyl acetate, butyl benzoate, or ethyl benzoate. In each case, spherical microcapsules are observed that are colloidally stable during storage, and mechanically stable during handling. The size bar applies to all four images in this figure.

FIG. 3 shows optical micrographs of polyurea capsules formed from Mondur ML and DETA, with 10% 1-dodecanol and 90% total co-solvent mixture, after storage in aqueous suspension for six months. The capsules formed using propyl acetate/DMP (10%/80%), butyl acetate DMP (10%/80%) and butyl acetate/DMP (20%/80%) all show spherical shape with no evidence for aggregation. The size bar applies to all three images in this figure.

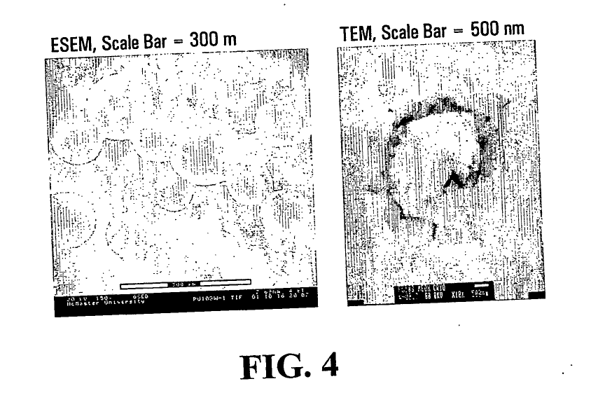

FIG. 4 shows environmental scanning electron microscopy (ESEM) and transmission electron microscopy (TEM) images for polyurea capsules formed from Mondur ML and DETA, with 20% 1-dodecanol and 80% butyl benzoate. These capsules show spherical shape similar to those capsules formed in absence of 1-dodecanol (not shown). The TEM image shows sections of the thin and fairly smooth capsule walls, in agreement with the low Mondur ML loading of 2.5%.

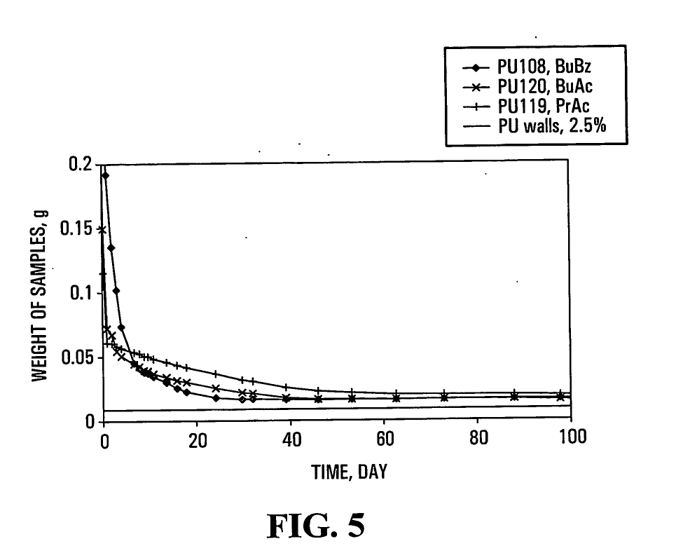

FIG. 5 shows the effect of using different single solvents, on the release from polyurea capsules formed from Mondur ML and DETA, with 20% 1-dodecanol and 80% solvent in the core. The three solvents used were butyl benzoate, butyl acetate and propyl acetate. In the case of propyl acetate, rapid release is observed during the initial period, corresponding to the low boiling point of propyl acetate, followed by a slow release for about 60 days. In the case of butyl acetate, a similar release profile is observed, though the transition from fast to slow release is less distinct compared with the case of propyl acetate. In the case of butyl benzoate, the transition from rapid to slow release is even more gradual, in agreement with the higher boiling point of butyl benzoate. In the case of butyl benzoate, the total release is faster than in the case of butyl acetate, and much faster than in the case of propyl acetate. It is hence suggested that the higher boiling solvent, butyl benzoate, remains in capsules longer than the lower boiling solvents, and hence can facilitate the release of the 1-dodecanol for a longer period of time.

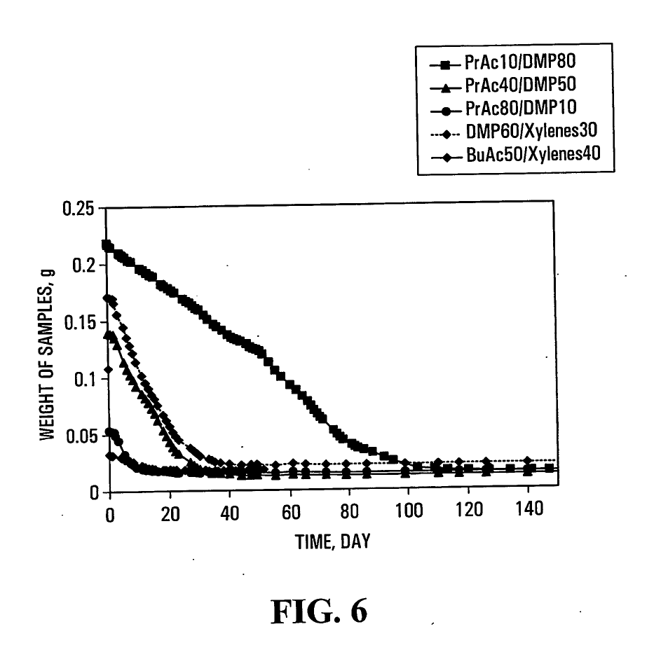

FIG. 6 shows the effect of co-solvent composition on release from polyurea microcapsules formed from Mondur ML and DETA, with 10% 1-dodecanol and 90% total co-solvent mixtures in the core. The co-solvent mixtures shown here are based on DMP and Xylenes, with co-solvents chosen to reduce or increase the total solvent polarity, respectively:
(i) butyl acetate 50%, xylenes 40%;
(ii) xylenes 30%, dimethyl phthalate 60%;
(iii) propyl acetate 80%, dimethyl phthalate 10%;
(iv) propyl acetate 40%, dimethyl phthalate 50%;
(v) propyl acetate 10%, dimethyl phthalate 80%.

As FIG. 6 shows, for the three DMP-PrAc co-solvent systems, nearly linear release profiles were observed. The length of the release period varies from about 30 to 100 days as the PrAc fraction changes from 80 to 10%. DMP-BuAc co-solvent systems have similar results.

When xylenes were used as a co-solvent, the weight of the residual samples levelled off at a slightly higher level, suggesting incomplete release. This is attributed to the poorer match of the properties of fill and polyurea even though the other co-solvent (DMP or BuAc) has already improved this property match.

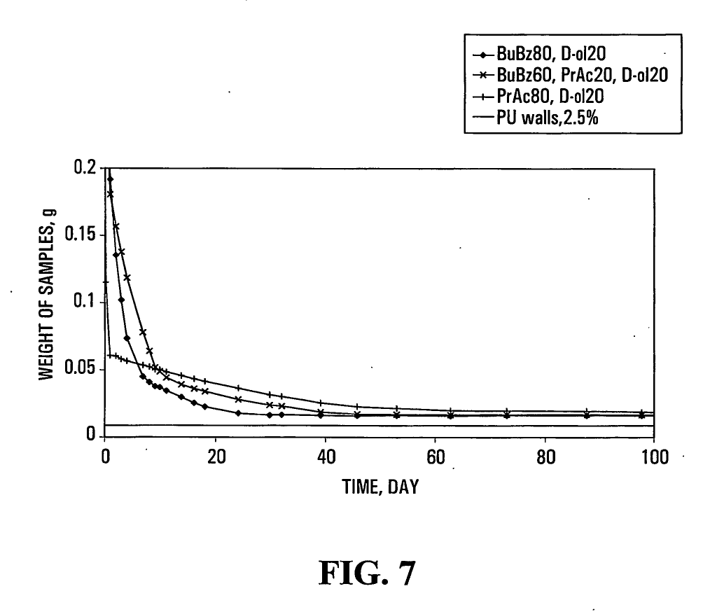

FIG. 7 shows graphically results of the effect of using different water-immiscible phases on release from microcapsules formed from Mondur ML/DETA, with 20% 1-dodecanol and 80% total co-solvent. The other components of the water-immiscible liquid were butyl benzoate (80%), butyl benzoate (60%) plus propyl acetate (20%) and propyl acetate (80%) respectively. The results demonstrate again that one can effectively adjust the release period by simply changing the co-solvent composition in the organic phase solvents. The addition of propyl acetate to the butyl benzoate slows down the fill release due to the poorer solvent properties for the polyurea, i.e., the greater difference between propyl acetate and polyurea in solubility parameter, as compared with the difference between butyl benzoate and polyurea.

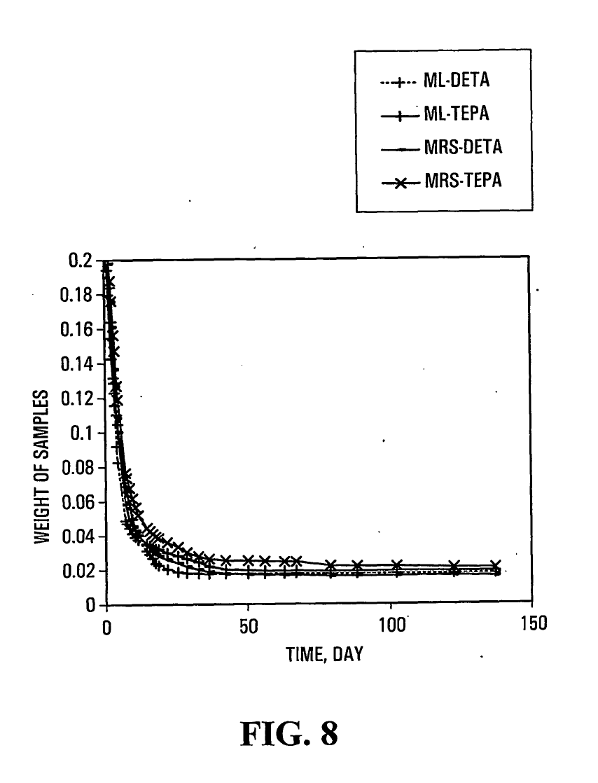

FIG. 8 shows graphically results of comparative tests using different isocyanates, which lead to different polyurea wall characteristics. There was used a water-immiscible fill mixture composed of butyl benzoate as solvent (80%) and 1-dodecanol (20%) as pheromone model, the isocyanate loading being 2.5%. Mondur ML has two isocyanate moieties per molecule, whereas Mondur MRS is a mixture of difunctional and several higher functional isocyanates, with on average between of 2.3-2.6 isocyanate moieties per molecule, so opportunity for crosslinking is greater with Mondur MRS.

The amines used were DETA and TEPA. DETA is considered to act mainly as a di-functional amine, with only limited crosslinking through the secondary amine in the centre of the molecule. TEPA is considered to give comparatively more crosslinking through the secondary and additional primary amines in the centre of the molecule.

Results of release of fill over time, are shown in FIG. 8 and show that release period is increased when the degree of crosslinking in the polyurea wall is greater. The capsules formed from Mondur ML/DETA release completely by about 100 days. Similar effects of crosslinking on release were observed when DMP-acetate (butyl and propyl) co-solvent systems were used.

In contrast to the experiments whose results are shown in FIG. 8, when xylene or DMP was used as solvent in an attempt to encapsulate 1-dodecanol at 10% loading, no stable microcapsules formed; initially formed capsules coagulated shortly after their formation.

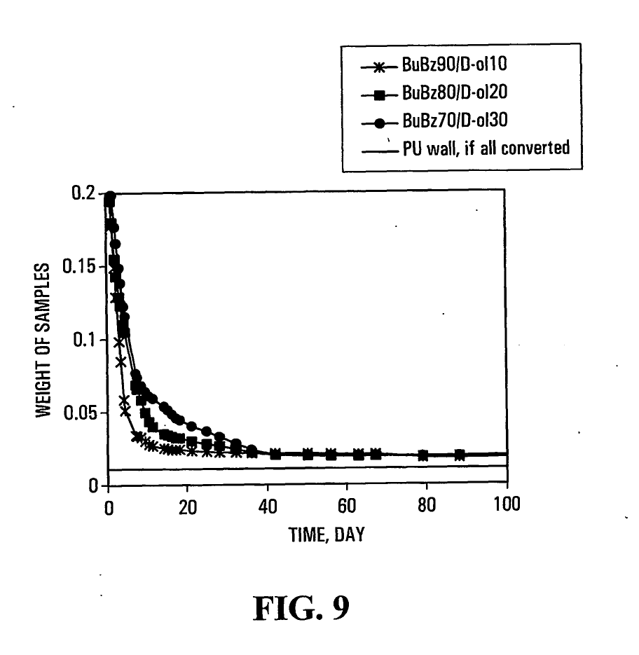

FIG. 9 shows results on release of varying the amount of 1-dodecanol encapsulated. Mondur ML at 2.5% loading and TEPA were used. The fills were mixtures of 1-dodecanol and butyl benzoate. It is noteworthy that by selection of appropriate water-immiscible phase the inventors were able to achieve a 30% loading of pheromone, and also that the microcapsulation yielded stable microcapsules that released the pheromone over a period of more than 30 days. The effect of 1-dodecanol loading is significant. The increase of loading from 10% to 30% led to an increase in the release period from about 10 days to more than 30 days. Much of the weightless during the first approximately five to ten days can be attributed to loss of solvent, butyl benzoate, while the release of the dodecanol dominates the weight loss during the latter stages of release.

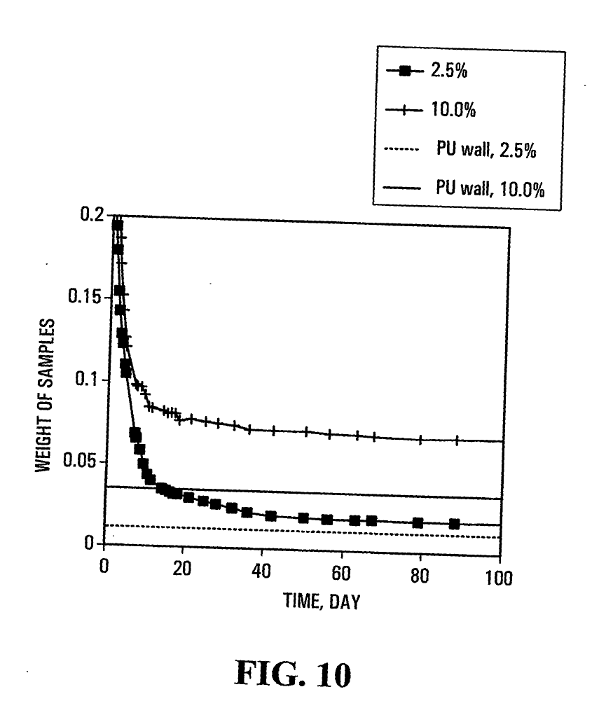

FIG. 10 shows results of experiments in which the isocyanate loading was varied. Mondur ML was used at 2.5% and 10% loading, with DETA. The fill was 20% 1-dodecanol and 80% butyl benzoate. It can be seen that higher isocyanate loading slightly extends the release period, but also significantly slows the release of the dodecanol and leads to retention of large amounts of fill even after 100 days of release.

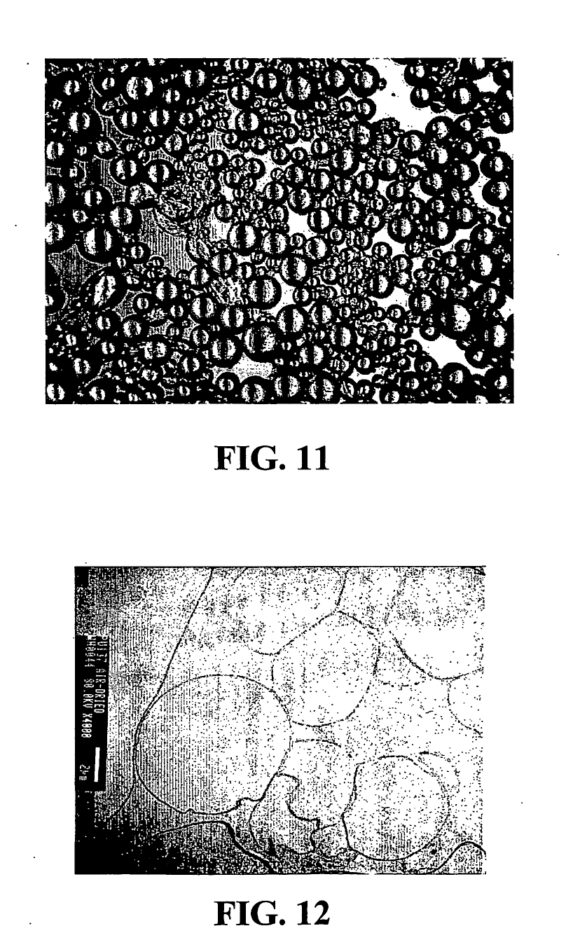

FIG. 11 shows optical micrographs of polyurea microcapsules formed from Mondur MRS and tetraethylenepentamine (TEPA). The oil phase consisted of 20 mL 1-dodecanol, 40 mL isopropyl myristate and 40 mL methyl isoamyl ketone (MIAK) and 2.5 g Mondur MRS. The aqueous phase consisted of 300 mL distilled water containing 0.1% polyvinyl alcohol (PVA) and 0.5 mL (0.54 g) Tween 80 surfactant. The capsules are formed by emulsifying the combined oil phase in 250 mL of the aqueous phase for 5 minutes at 400 rpm, adding TEPA dissolved in the remaining 50 mL aqueous phase, and reducing the stirring speed to 250 rpm one minute after adding the TEPA. The capsules show spherical shape. Mondur MRS is less soluble in isopropyl myristate than the lower molecular weight analog Mondur ML. As a result, some of the isopropyl myristate has been replaced with the more polar methyl isoamyl ketone in this example. The mixture of isopropyl myristate, having a fairly low hydrogen bonding solubility parameter, and MIAK, having a high hydrogen bonding solubility parameter, is capable of dissolving both Mondur MRS and the pheromone to form a homogeneous organic phase. In addition, this solvent mixture is capable of swelling the polyurea wall sufficiently to permit both in-diffusion of the amine during capsule formation, and release of the fill during the release period. An additional advantage of this composition is that both isopropyl myristate and MIAK are approved for agricultural use in the United States.

FIG. 12 shows a transmission electron micrograph (TEM) of the polyurea capsules formed from Mondur ML and DETA, using 20% 1-dodecanol and 80% isopropyl myristate for the organic phase. The TEM shows the thin, dense wall formed at the interface between the aqueous and organic phases. Isopropyl myristate is a branched alkyl ester or a long chain aliphatic acid. Its Hansen hydrogen-bonding and polarity parameters are near the lower end of the range acceptable to achieve sufficient swelling of aromatic polyurea shells.

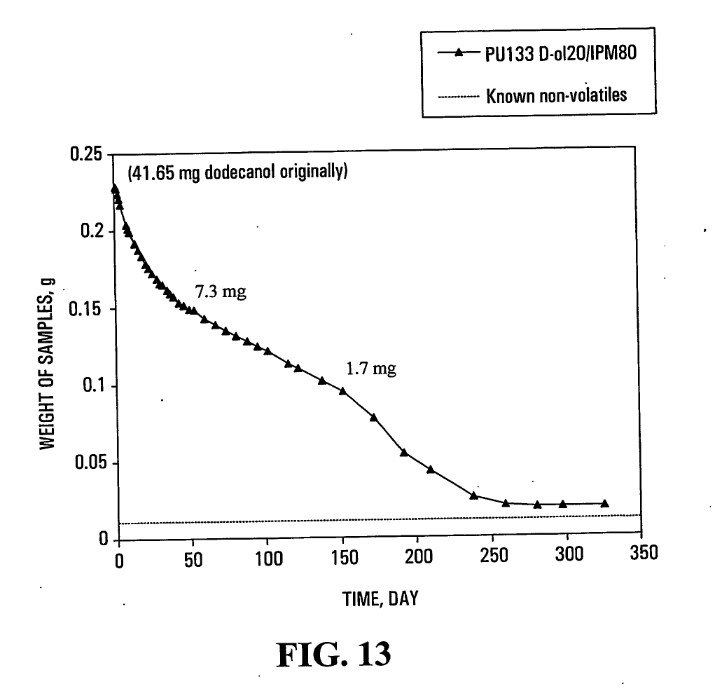

FIG. 13 shows the results of observations of release rates from polyurea capsules described in FIG. 12, formed with 20% 1-dodecanol and 80% isopropyl myristate and using Mondur ML and DETA. The graph reflects the results of weight loss measurements. The numerical values along the graph indicate the amount of 1-dodecanol remaining in the capsules at the indicated times. These data indicate that release of 1-dodecanol is substantially complete after 150 days. These data also indicate that in cases such as this, where the solvent has a significantly higher boiling point compared with the pheromone, release of the pheromone is still effective, as sufficient solvent is present to swell the polyurea wall during the release phase.

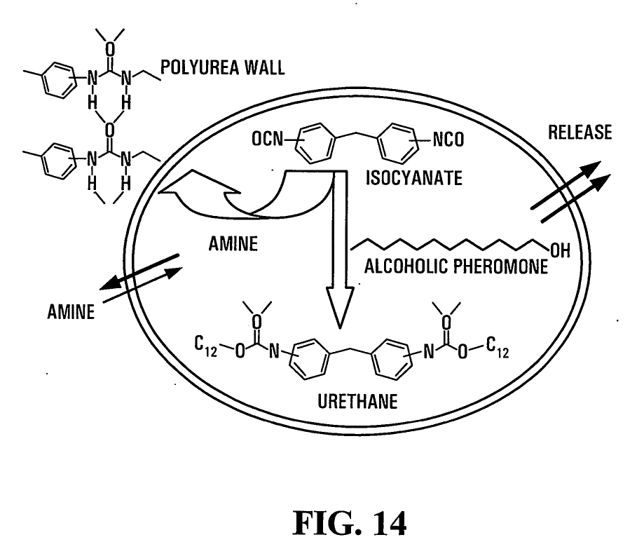

FIG. 14 illustrates how the in-diffusing amine and oil-borne hydroxy-functional pheromone compete for the available isocyanate in each forming capsule. The undesired urethane-forming side-reaction can be minimized by using core-solvents that by nature of their hydrogen-bonding ability and polarity can both physically swell the forming polyurea, and facilitate partitioning of the amine into the organic phase. In addition, it is helpful if the core-solvents have boiling points close or higher than that of the pheromone, in order to be able to swell the polyurea wall during the release period. It is further helpful to reduce the isocyanate and pheromone loadings in the core to 2.5% and 20%, respectively.

In addition to the experiments summarized in the figures, polyurea capsules based on Mondur ML and DETA, as well as Mondur MRS and TEPA, can also be formed using polar, less volatile esters such as triglycerides. Specifically, stable polyurea capsules were formed from Mondur ML and DETA, with 20% 1-dodecanol and 80% glycerol tributyrate in the core. Similar capsules may also be formed using glycerol tributyrate or other triglycerides, in conjunction with other solvents.

As stated above, attempts to encapsulate 1-dodecanol at 10% loading in DMP, alone did not result in formation of stable microcapsules. In experiments with solvents of lower polarity than DMP success was achieved. Thus success was achieved with dibutyl phthalate (DBP) (90%) and 1-dodecanol (10%). Success was also achieved with microcapsules of Mondur ML at 2.5% loading and DETA with fills of propyl acetate (80%) plus 1-dodecanol (20%) and of butyl acetate (80%) plus 1-dodecanol (20%), as well as with fills of ethylbenzoate (80%) and 1-dodecanol (20%) and with butyl benzoate (80%) and 1-dodecanol (20%). Results of weight loss measurements as an indicator of fill release for some of these cases are shown graphically in FIG. 5.

Encapsulation of 1-dodecanol with butyl benzoate as solvent was successful at loadings of 10%, 20% and 30%, using Mondur ML at 2.5% loading and TEPA, and results are shown in FIG. 9. Encapsulation attempts with ethyl benzoate as sole solvent were successful, but those with methyl benzoate as sole solvent were unsuccessful and the microcapsules coagulated during the last stages of reaction. It is believed that methyl benzoate is too polar and that admixture with a co-solvent to reduce polarity somewhat would enable it to be used successfully.

While DMP can not be used as a single solvent in the encapsulation of 1-dodecanol, DMP with a small amount of less polar co-solvent works well for this purpose. DMP/BuAc and DMP/PrAc, with the co-solvent ratio ranging from 1/8 to 8/1 and containing 1 part (10%) 1-dodecanol, were tested. Similarly, DMP/xylenes and BuAc/xylenes at co-solvent ratio up to 5/4 were also tested, again with 1 part (10%) dodecanol. Stable capsules were observed in each case. However, the capsules prepared using xylenes as a co-solvent tend to coagulate during storage, and this tendency increases with increasing xylene fraction.

The invention reveals that in the encapsulation of reactive materials, such as 1-dodecanol, the properties of the organic phase in terms of polarity, hydrogen bonding ability, and boiling point are very important for the formation of stable capsules. Adjusting the properties of organic phase can be realized by either choosing a suitable solvent or by using a co-solvent.

Butyl benzoate is a good choice as a single solvent to prepare polyurea capsules encapsulating 1-dodecanol. It has good mutual solubility with 1-dodecanol, and a similar solubility parameter to that of polyurea. The capsules have reasonably good stability, and have a release period of about 10 to 30 days when using Mondur ML and DETA to form polyurea capsules with a Mondur loading of 2.5 (w/v) to the organic phase.

Alkyl acetates also have good mutual solubility with 1-dodecanol, however, propyl or butyl acetates evaporated fast at the beginning, leave 1-dodecanol behind for a slow and possibly incomplete release.

DMP-acetate co-solvent systems are a good choice for the encapsulation of 1-dodecanol as regards the stability of the capsules, nearly linear release profiles, and the adjustable release period. The release period varies from about 30 to 100 days as PrAc fraction changes from 80 to 10%.

Isopropyl myristate, and mixtures of isopropyl myristate with methyl-isoamyl ketone, represent organic phases that fulfill the requirements for sufficient hydrogen-bonding and polarity, and are accepted for use in agricultural situations. The high boiling point of isopropyl myristate additionally ensures that it will be present in the capsules during the release period to swell the capsules and facilitate release.

The microcapsule suspension as obtained from the interfacial reaction still contains residual amounts of stabilizer and/or surfactant. It was observed that washing the capsules with water to remove most of this residual stabilizer and/or surfactant resulted in increased release rates, and more complete release over time. This is possibly due to the residual stabilizers and/or surfactants forming a hydrophilic layer on the outside of the capsules, that is responsive to humidity and acts as an additional release barrier to the hydrophobic fill.

All publications, patents and patent applications cited in this specification are herein incorporated by reference as if each individual publication, patent or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

It must be noted that as used in this specification and the appended claims, the singular forms “a”, “an”, and “the” include plural reference unless the context clearly dictates otherwise. Unless defined otherwise all technical and scientific terms used herein have the same meaning as commonly understood to one of ordinary skill in the art to which this invention belongs.

1. A process for encapsulation of a hydrophobic organic molecule in a polyurea microcapsule by interfacial polymerization, the process comprising contacting
c) an aqueous phase comprising an amine-bearing compound selected from a diamine and a polyamine, and
d) a water-immiscible phase comprising a water-immiscible solvent, an isocyanate-bearing compound selected from a diisocyanate and a polyisocyanate, and a hydrophobic organic molecule
wherein the water-immiscible solvent has a solubility parameter that is below the solubility parameter of the polyurea microcapsule.

2. The process according to claim 1, wherein the solubility parameter of the water-immiscible solvent is within the range of 3 to 8 Mpa^{1/2} of the solubility parameter of the polyurea microcapsule.

3. The process according to claim 1 or 2, wherein the polyurea microcapsule is swollen by the water-immiscible solvent.

4. The process according to any one of claims 1 to 3, wherein the water-immiscible solvent has a boiling point that is lower than the boiling point of the hydrophobic organic molecule.

5. The process according to claim 4 wherein the boiling point of the water-immiscible solvent is within 60° C. of the boiling point of the hydrophobic organic solvent.

6. The process according to any one of claims 1 to 5, wherein the water-immiscible solvent is comprised of two or more solvent components, and wherein the boiling point of one of the solvent components is within 20° C. of the boiling point of the hydrophobic organic solvent.

7. The process according to any one of claims 1 to 6, wherein the hydrophobic organic molecule is volatile.

8. The process according to any one of claims 1 to 7, wherein the hydrophobic organic molecule is a pheromone.

9. The process according to claim 8, wherein the pheromone comprises a functional group selected from hydroxyl, epoxy, aldehyde and ester.

10. The process according to any one of claims 1 to 6, wherein the hydrophobic organic molecule comprises a compound that is selected from the group comprising a mercaptan, an essence of garlic, putrescent eggs, capsaicin, a perfume, a pharmaceutical, a fragrance, a flavouring agent, a pigment, a dye, an antioxidant, a light stabilizer, and a UV absorber.

11. The process according to any one of claims 1 to 6, wherein the hydrophobic organic molecule is selected from an E/Z-11 C₁₄ aldehyde, a Z-10 C₁₉ aldehyde, a Z-11 C₁₄ acetate, a Z-8 C₁₂ acetate, an E,E-8,10 C₁₂ alcohol, E or Z 7-tetradecen-2-one, 4-allylanisole, E-11-tetradecen-1-ol, a Z-11 C₁₄ alcohol, a Z-8 C₁₂ alcohol, an E,E-8,10 dodecadiene-1-ol alcohol, linalool, terpineol, fenchone, a keto-decenoic acid, a hydroxy-decenoic acid, 4-allylanisole, and 7,8-epoxy-2-methyloctadecane.

12. The process according to any one of claims 1 to 11, wherein the water-immiscible solvent comprises one or more of a linear or branched C₁-C₁₂ alkyl ester or diester of acetic acid, propionic acid, succinic acid, adipic acid, benzoic acid or phthalic acid.

13. The process according to any one of claims 1 to 11, wherein the water-immiscible solvent comprises a linear or branched C₁-C₁₂ triester of glycerol, or a C₁-C₁₂ diester of ethylene glycol, propylene glycol or butylene glycol.

14. The process according to any one of claims 1 to 11, wherein the water-immiscible solvent comprises a linear or branched C₁-C₁₂ ester of a linear or branched aliphatic acid having between 1 and 16 carbons.

15. A microcapsule comprising a water-immiscible solvent and a hydrophobic organic molecule, encapsulated by a polyurea microcapsule which is swollen by the water-immiscible solvent.

16. The microcapsule according to claim 15, wherein the water-immiscible solvent has a solubility parameter that is below the solubility parameter of the polyurea microcapsule.

17. The microcapsule according to claim 16, wherein the solubility parameter of the water-immiscible solvent is within the range of 3 to 8 Mpa^{1/2} of the solubility parameter of the polyurea wall.

18. The microcapsule according to any one of claims 15 to 17, wherein the hydrophobic organic molecule is present in an amount greater than 5%, based on the weight of the water-immiscible solvent.

19. The microcapsule according to any one of claims 15 to 17, wherein the hydrophobic organic molecule is present in an amount greater than 10%, based on the weight of the water-immiscible solvent.

20. The microcapsule according to any one of claims 15 to 17, wherein the hydrophobic organic molecule is present in an amount greater than 20%, based on the weight of the water-immiscible solvent.

21. The microcapsule according to any one of claims 15 to 17, wherein the hydrophobic organic molecule is present in an amount greater than 30%, based on the weight of the water-immiscible solvent.

22. The microcapsules according to any one of claims 15 to 21, wherein the hydrophobic organic molecule is volatile.

23. The microcapsules according to any one of claims 15 to 22, wherein the hydrophobic organic molecule is a pheromone.

24. The microcapsule according to claim 23, wherein the pheromone comprises a functional group selected from hydroxyl, epoxy, aldehyde and ester.

25. The microcapsules according to any one of claims 15 to 21, wherein the hydrophobic organic molecule comprises a compound that is selected from the group comprising a mercaptan, an essence of garlic, putrescent eggs, capsaicin, a perfume, a pharmaceutical, a fragrance, a flavouring agent, a pigment, a dye, an antioxidant, a light stabilizer, and a UV absorber.

26. The microcapsules according to any one of claims 15 to 21, wherein the hydrophobic organic molecule is selected from an E/Z-11 C₁₄ aldehyde, a Z-10 C₁₉ aldehyde, a Z-11 C₁₄ acetate, a Z-8 C₁₂ acetate, an E,E-8,10 C₁₂ alcohol, E or Z 7-tetradecen-2-one, 4-allylanisole, E-11-tetradecen-1-ol, a Z-11 C₁₄ alcohol, a Z-8 C₁₂ alcohol, an E,E-8,10 dodecadiene-1-ol alcohol, linalool, terpineol, fenchone, a keto-decenoic acid, a hydroxy-decenoic acid, 4-allylanisole, and 7,8-epoxy-2-methyloctadecane.

27. The microcapsule according to any one of claims 15 to 26, wherein the water-immiscible solvent has a boiling point which is lower than that of the hydrophobic organic molecule.

28. The microcapsule according to claim 27, wherein the boiling point of the water-immiscible solvent is within 60° C. of the boiling point of the hydrophobic organic molecule.

29. The microcapsule according to any one of claims 15 to 28, wherein the water-immiscible solvent is comprised of two or more solvent components, and wherein the boiling point of one of the solvent components is within 20° C. of the boiling point of the hydrophobic organic solvent.

30. The microcapsule according to any one of claims 15 to 29, wherein the water-immiscible solvent comprises one or more linear or branched C₁-C₁₂ alkyl esters or diesters of acetic acid, propionic acid, succinic acid, adipic acid, benzoic acid, and phthalic acid.

31. The microcapsule according to any one of claims 15 to 29, wherein the water-immiscible solvent comprises a linear or branched C₁-C₁₂ triester of glycerol, or a C₁-C₁₂ diester of ethylene glycol, propylene glycol or butylene glycol.

32. The microcapsule according to any one of claims 15 to 29, wherein the water-immiscible liquid comprises a linear or branched C₁-C₁₂ ester of a linear or branched aliphatic acid having between 1 and 16 carbons.

33. Use of a microcapsule as claimed in any one of claims 15 to 32, for the controlled release of a volatile hydrophobic organic molecule.
